(19) United States
(12) Patent Application Publication
(10) Pub. No.: US 2004/0214891 A1
(43) Pub. Date: Oct. 28, 2004

(54) Method for inhibiting pathogenic bacteria and fungi growth and microbicidal composition

(76) Inventors: Janusz Marcinkiewicz, Krakow (PL); Andrzej Kasprowicz, Krakow (PL)

Correspondence Address: Mr. Miroslaw Paczuski,, PA TRADE Int'l, P.O. Box 76, Center Valley, PA, 18034, US

(21) Appl. No.: 10/825,288

(22) Filed: Apr. 16, 2004

(30) Foreign Application Priority Data
Apr. 22, 2003 (PL) P - 359792
Apr. 7, 2004 (PL) P - 367052

(51) Int. Cl. A61K 31/185

(52) U.S. Cl. 514/553

(57) ABSTRACT

In a method for inhibiting pathogenic bacteria and fungi growth, bacteria and fungi are treated with an effective quantity of taurine bromamine [TauBr]. The effective quantity of taurine bromamine [TaurBr] has concentrations of 10-100% content by weight.

The microbicidal composition, according to the invention, contains taurine bromamine [TauBr] being its essential component at concentrations of 10-100% by weight.

### CROSS-REFERENCE TO RELATED APPLICATIONS

This application claims priority to Polish Applications No. P 359792, filed Apr. 22, 2003 and No. P 367052 filed Apr. 7, 2004, the contents of which are incorporated herein by reference.

### BACKGROUND OF THE INVENTION

1. Field of the Invention

The present invention relates to a method for inhibiting pathogenic bacteria and fungi growth and a bactericidal composition to be applied in the treatment of dermal diseases and associated diseases attributable, among others causative factors to propionibacterium types, particularly in the treatment of common acne (Acne vulgaris) as well as a composition comprising taurine, and its uses in filed of cosmetics and/or pharmacology, especially dermatology.

2. Brief Description of the Background of the Invention Including Prior Art

The human skin and the external mucosa are constantly in contact with their surrounding environment. Hence they are exposed to diseases caused by bacteria, fungi and viruses. Bacterial skin diseases are mostly caused by mixed staphylococcal/streptococcal infections. Dermatomycoses are pathogen fungi. One of the most common skin diseases is acne in which bacteria Propionibacterium acnes is an etiological factor. The acne (Acne vulgaris) is experienced during pubertal changes, this condition being associated with the development of hair-follicles, together with their associated sebaceous and excretory duct systems in association with the formation of keratinocytes. Despite the progress in diagnostic techniques, the acne remains a problem, not only for patients, but also for medical diagnostic teams attempting to alleviate the problem. It is assumed that some 85% of the human population experiences these changes, associated with maturation, in a more or less intense form. More aggravated symptoms, at varying levels, occur within the 14-18 year-old age-range, and involve some 10% of the population. Either gender is susceptible to the condition, but it is particularly prevalent in its severest form for pubertal males, due to genetic and hormonal changes.

The etiology of acne is attributable to several causative origins, including; hormonal balances, genetics, immune reactions, and bacterial factors, among others to be mentioned. Juvenile acne occurs as a result of the increase of sebum production by the cutaneous glands under the influence of increased secretion of androgenic hormones during the pubertal phase. In the development of the acne, four factors have primary significance: increased secretion of sebum, excessive keratosis, blockage of the folicular/sebacial duct outlets and invasion and colonization by anaerobic bacteria (Propionibacterium acnes) that cause an inflammatory state in and around the sebaceous glands and result in the formation of so-called blackheads, inflamed papulae, surface cysts, atheromatous cysts and pustules. Androgens, as already mentioned, stimulate the secretion of sebum from the sebaceous glands. Sebum is a fatty substance that maintains a proper level of hydration of the skin and in keratin, the latter constituting the principal component of hair. The other main causative factor for the progenesis of acne is the obstruction of the ducts that exude sebum onto the surface of the skin. This leads to enlargement of the ducts of the sebaceous glands and the development firstly of closed comedones, and then to their bursting. The blockage results in the excessive amounts of generated sebum being retained. Blackheads or points with white heads appear. These changes are non-inflammatory in character. If the walls of the overfilled follicle burst, the secretions may be exuded into adjacent tissue-layers, resulting in the development of inflammatory acne. The infection invades the dermal tissue, cysts arise and these may burst at a later stage, leaving transient or even persistent tissue scarring. These changes may become subject to super-infection. The bacterium P. acnes, by introducing chemotactic factors, may engender a polynuclear leukocytes inflow to the sebaceous glands. As a result of the leukocytes induced phagocytes of the bacteria, hydrolytic enzymes are liberated, leading to the destruction of the walls of the sebaceous glands, allowing their content to invade the corium and leading to the development of an inflammatory condition. Papulo pustuar eruptions may appear. In severe forms of acne, deep, purulent infiltrations and fistulae are generated, leading to local scarring. Within the blocked sebaceous glands the development of anaerobic bacteria takes place. These bacteria produce enzymes that decompose the sebum into free fatty acids, thereby intensifying the inflammatory state. The bacteria P. acnes and P. granulosum are mainly found in areas of the human body rich in sebaceous glands such as the head, the chest, the back, mainly in the interscapular region. The above mentioned factors, associated with a raised concentration of microorganisms, give rise to chronic inflammatory processes, involving the sebaceous hair follicles and the sebaceous glands with which they are associated. Stimulation of the sebaceous glands induces increased production of sebum whose lipid-rich secretions form an excellent medium in which bacteria may breed. A correlation between the severity of the degree of acne and the level of secretion of sebum has been observed. At the same time as the process is taking place, dyskeratinisation occurs—i.e. an abnormal proliferation of ductal keratomycetes and the avulsion of sebaceous gland cells, leading to the blockage of the ducts, thereby creating ideal conditions for the development of anaerobic bacterial growths.

In recognition of the variable etiology of changes associated with acne, the potential increase in the intensity of such changes and the various sites in which the condition may occur, often diverse suggestions have been made as to how to treat this pathological state. The principal aim of treatment for common acne is to arrest the symptoms of seborrhea by constricting the dermal pores, removal of the retained secretions, prevention of the occurrence of comedones, reducing the secretion of sebum, alleviating inflammatory states and decreasing hypersensitivity. Various therapeutic regimes have been applied, such as the administration of hormones (estrogens and pro-estrogens), courses of vitamins (B2, A, C, E, PP) or antibiotics (tetracycline, erythromycin). The traditional treatment uses local applicants containing dehydrative compounds with the addition of salicylic acid, menthol and sulphur, recorcin, exposure to sunlight or UV light (the latter no longer being practiced). The use of salicylic acid is also no longer recommended for the alleviation of the symptoms of acne due to its irritant effect in many cases. As an adjunct to treatment, large doses of vitamins are recommended, particularly those belonging to the vitamin B group. Hormone-based medicines form one of other groups; those which diminish the effect of androgens. Medicines containing progesterones are most frequently prescribed. They are most effective in cases of mild acne. Their action causes a diminution in concentrations of androgens and thereby reduces the rate of sebum secretion and the component creating conditions for inflammation. Spironolactone is another medicine with a similar effect and is popular for the treatment of acne. It has also been demonstrated that corticosteroids given generally, inhibit the secretion of androgens. Corticosteroids are mainly administered by direct injection to isolated deforming acne changes of a cystic or tubercular character to accelerate their rate of healing. Nevertheless, hormone-based medicines are not the most suitable and are only partially effective in cases exhibiting advanced dermal pathological conditions. Additionally, if large doses of Spironolactone (as mentioned earlier) are administered, women may experience menstrual irregularities and men may suffer from gynaecomastia and loss of libido. Dizziness and a general feeling of fatigue may also be experienced.

Retinoids form another group of medicines, being derivatives of Vitamin A. They are applied to slow the process of keratinisation, and are typically applied both locally and generally. Isotretynoine is characterized by its effect in stabilizing abnormal keratosis pilaris and inhibiting the process of desquamination of epithelial cells by weakening their interconnections. Among generally applied Retinoids, Isotretynoine has been proved to be the most effective. Its effectiveness relies on its reducing sebum secretion. Simultaneously it normalizes the process of keratinisation in the hair follicles and although it has no direct antibacterial activity its effects, as noted above, result in a decrease in the changes occurring within hair follicles. Treatment with Isotretynoine is particularly effective in severe cases of acne that exhibit inflamed cysts with the proviso that such treatment should be carefully monitored, especially in the case of female patients as it is not recognized as being entirely safe. Such patients should be tested for pregnancy before treatment is commenced, least fetal damage be caused. The treatment is contraindicated in cases of pregnancy. It is also recommended that a course of treatment with Isotretynoine should be accompanied by the use of oral contraceptives by women. The effect of Retinoids is the normalization of the desquamation process in the outlet ducts of the sebaceous glands, thereby facilitating the exudation of gland secretions and thereby resulting in a discernible reduction of comedogenesis. For this reason their main application is in cases of acne comedonica. Contraindications are also exhibited by exsiccation, desquamation and reddening of the skin, these being frequent symptoms in patients taking Isotretynoine. The use of such medicines may cause severe undesirable side-effects, particularly teratogenic reactions. Medicines from the Retinoids group, as noted earlier, have no direct bactericidal activity. Such action is desirable in cases requiring treatment for dermal changes. In addition to the adverse effects of therapy using Retinoids, there are observed increases in levels of transaminases, bilirubin and hyper-three-glycerids may occur. For this reason patients in the risk-group i.e. with a family history of hyper-three-glyceridemia or diabetes are advised to be examined both before treatment and two months thereafter, not only for general medical examination but with particular attention being paid to the liver and to lipid levels. Frequently-experienced side effects also include drying of the mucosae of the lips and the nasal cavity and conjunctivitis.

Yet another group of medicines used to treat pathological dermal states are antibiotics. They are normally used for local lavage or are applied locally as gels or ointments as well as being taken orally. The main activity of such medicines lies in their antibacterial effect, thereby diminishing the level of colonization by bacteria of the external pores of the sebaceous glands. By decreasing the number of micro-organisms they directly decrease concentrations of free fatty acids, thereby lowering the levels of lipase and protease in the hair follicles. Hence antibiotics are both antibacterial and anti-inflammatory in their action. The main groups of antibiotics used for acne treatment are tetracyclins, macrolids, clindamycin and cotrimoctrisol. The main disadvantage of antibiotics is that they cannot be used for extended periods, their application being restricted to 3-4 weeks. Moreover, the excessive, uncontrolled and indiscriminate use of antibiotics and sulphonamides, as was the case in the 1970s and '80s has resulted in the fact that nowadays the use of many chemotherapeutic agents even on the skin has resulted in bacterial immunity to them, thereby rendering such agents ineffective. This concerns, among others, neomycin and tetracycline. There is also the possibility of adverse reactions.

Apart from antibiotics a number of locally-applied agents are used for the treatment of mild acne. These include benzyl peroxide, azelan acid, sulphur preparations and salicylic acid, the latter having been mentioned earlier.

A lesser-known therapy is to inject triamcinolon, a type of steroid directly into the cyst. This may have the temporary side-effect of darkening the skin around the site of the injection.

Adjunctive therapies are also used and these should be preceded by the patient being clearly and comprehensibly informed about them. Individual approach of patients' regimes, possibly administering several medicines simultaneously, in conjunction with hygiene/cosmetic procedures, and if necessary with cryotherapy, lasertherapy or treatment with liquid nitrogen may alleviate the symptoms of acne considerably.

Clinical tests have also been conducted using phototherapy. The promising effects of photodynamic treatment observed after the external use of ALA solution and irradiation of the skin of patients with acne are primarily a decrease in the number of inflammatory changes, the photocytotoxic damage to the sebaceous glands the long-term suppression of sebum production and a considerable decrease in the numbers of bacteria.

In some cases vaccines and autovaccines are used. Irrespective of which is applied an immune reaction occurs identically because all these preparations contain Propionibacterium acnes strains with strong immunomodulating activity. The practice indicates, however, that in cases of patients treated with vaccines or autovaccines, there is a need to simultaneously administer preparations with antibacterial and anti-inflammatory activity. On the other hand, it has been stated that treatment with vaccines containing antigens Propionibacterium acnes and Propionibacterium granulosum turned out to have little effect—only about 30% of patients experienced a limited improvement and the same proportion of patients showed a worsening of their condition. Administering autovaccines, in the light of contemporary knowledge, would seem to be unjustified.

Another treatment of acne is known from the U.S. Pat. No. 4,772,592 that describes a water-in-oil emulsion suitable for topical application to human skin. The water-in-oil emulsion comprises in addition to water a C.sub.1 to C.sub.4 alkyl lactate, a silicone oil ingredient containing a dispersion in a volatile siloxane of a polymer of dimethyl polysiloxane with polyoxyethylene and/or polyoxypropylene side chains. These substances are present in specified quantities. Volatile polar liquids, such as alkyl lactate and alkanol constitute essential components of the emulsion because, in association with silicone oil they act to stabilize it. However, the C.sub.1 to C.sub.4 alkyl lactate irritates the skin and the mucosae.

### SUMMARY OF THE INVENTION

Purposes of the Invention

It is an object of this invention to provide a method for inhibiting pathogenic bacteria and fungi growth that is more effectively than known methods.

It is another object of this invention to provide a bactericidal composition to be applied in the treatment of dermal diseases allowing selectively acting on pathogenic bacteria and fungi.

These and other objects and advantages of the present invention will become apparent from the detailed description, which follows.

Brief Description of the Invention

According to the invention the method consists of treating bacteria and fungi with a therapeutically effective amount of taurine bromamine [TauBr] (N-bromo taurine) or an effective quantity of taurine bromamine [TauBr]. The therapeutically effective amount of taurine bromamine [TauBr] or the effective quantity of taurine bromamine [TaurBr] has concentrations of 10-100% content by weight. The bactericidal composition, according to the invention, contains taurine bromamine [TauBr] being its essential component at concentrations of 10-100% by weight. The taurine bromamine [TauBr] with cetomakrogel emulsion or ethyl-cellulose gel or powder or glycerol can be used as a cosmetic or pharmaceutical composition or a medicine in treatment of pathological dermal conditions, especially in treatment of acne. The taurine bromamine [TauBr] can be a solution in an ethyl-cellulose gel or a cream or a powder with pH of 5-7.

The composition, besides the taurine bromamine, can contain talc in amount of 10.0-24.0% by weight, glycerol in amount of 14.0-40.0% by weight, distilled water in amount of 5.0-30.0% by weight and menthol in amount of 0.5-2.0% by weight or 5% methylcellulose solution in amount of 50.0-65.0% by weight or cetomakrogel emulsion in amount of about 15.0% by weight, liquid paraffin in amount of about 15.0% by weight, vaseline in amount of about 15.0% by weight and a mixture of preservative agents.

The composition, besides the taurine bromamine, can contain a lipophyllic phase—liquid paraffin, acetyl alcohol and an hydrophyllic phase—propylene glycol and estolate.

The taurine bromamine [TauBr] may be included as a main ingredient of soap used for disinfecting a human skin.

The novel features, which are considered to be characteristic for the invention are set forth in the appended claims. The invention itself, however, both as to its composition and its methods of application, will be best understood from the following description when read in connection with the accompanying examples.

### DESCRIPTION OF INVENTION AND PREFERRED EMBODIMENT

It is known that taurine chloramine [TauCl] (N-chloro taurine) has bactericidal properties. Its effect has been described in Hyg. Med. 18, 330-326, 1992. During the studies, it has been found, unexpectedly, that taurine bromamine [TauBr] (N-bromo taurine), which shows much stronger bactericidal activity than taurine chloramine [TauCl], produces a bactericidal effect on bacteria of Propionibacterium types, which are one of etiological factors of common acne (Acne vulgaris). Moreover, it turned out that taurine bromamine does not destroy the whole bacterial flora but acts selectively in certain conditions.

Different methods for producing taurine bromamine are known in present biochemical technology. In order to evaluate its bactericidal properties according to the invention, taurine bromamine was prepared on the day of the clinical test. In this order the solution of 180 mM NaOCl (Aldrich, Germany) was mixed in a phosphate buffer (POCH, Gliwice, Poland) to obtain NaCl solution with a concentration of 60 mM. The 60 mM solution of NaCl was mixed in equal proportions with a 100 mM solution of NaBr (POCH, Gliwice, Poland). NaOBr obtained in this way was diluted in a phosphate buffer (POCH, Gliwice, Poland) with a final concentration of NaOBr not exceeding 10 mM. To obtain taurine bromamine a solution of NaOBr was introduced by drops (whilst stirring very slowly) to the taurine solution (Sigma, St. Louis, Mo.) with a concentration of ca. 100 mM. The concentration of the taurine solution was 8-12 times higher than the concentration of the NaOBr solution, although taurine may be used in higher concentrations—up to 100 times greater. Preferably, while producing taurine bromamine, the concentration of the taurine solution was around 10 times higher than the concentration of the NaOBr solution. This resulted in the taurine bromamine solution having a molar concentration not exceeding 10 mM. The molar concentration of the taurine bromamine produced was assessed by measuring the extinction at a wave-length λₘₐₓ=286 nm, where the molarity of taurine bromamine equals value of extinction in peak 430⁻¹. To produce, for example 10 ml of 4-6 mM TauBr solution, 0.33 ml of 180 mM NaOCl in 0.67 ml of phosphate buffer (pH 7.4) was mixed with 1 ml of 100 mM NaBr. The NaOBr so obtained was dissolved in 3 ml of phosphate buffer and stirred very gently while 5 ml of 100 mM taurine was introduced in drops. The TauBr solution was tested at pH ranging from 5.0 to 8.0.

For research purposes, two bacterial strains commonly found on the human skin were chosen: Propionibacterium acnes (causing acne) and Staphylococcus epidermides (being a part of physiological flora of the skin). Selected microorganisms were lyophilized and kept at room temperature. To prepare strain cultures for immune research the lyophilisate was dissolved in 0.5 ml of 0.9% NaCl solution. The mixture was stirred and seeded on a growth medium. Incubation was conducted in anaerobic conditions at 37° C. for 48-72 hrs. A suspension of bacteria at a concentration of 5×10⁹ CFU/ml was then prepared.

In order to assess the microbicidal activity of the TauBr solution, the Proponibacterium acnes and the Staphylococcus epidermides bacteria were incubated in closed tubes with TauBr solution. The range of concentrations tested was 1-1000 μM. The incubation was conducted at room temperature for 30 minutes. When this process was completed, bacteria were seeded onto a growth medium. After an appropriate period of time the number of developed colonies were counted. Next, estimation of the susceptibility of the Proponibacterium acnes and Staphylococcus epidermides bacteria to the agent tested [TauBr] was determined. Analysis of the susceptibility of the strains tested proved the bactericidal activity of TauBr to the Proponibacterium acnes for which the inhibitory concentration was below 32 μM (MIC) and to the Staphylococcus epidermidis for which the inhibitory concentration was above 125 μM (MIC)

In further test, the microbicidal composition described in the example, in the form of an emulsion, was applied to the patient's skin. The microbicidal composition consisted of: an active substance—TauBr at concentrations of 10-50% and a base (emulsion)—made up to 100%. The base (emulsion) contains a 16% lipophyllic phase—liquid paraffin, acetyl alcohol and an 84% hydrophyllic phase—propylene glycol and estolate (or lauryl sulphate). The bactericidal formulation is particularly useful in the treatment of inflammatory skin conditions caused by bacterial or mycotic infections such as acne and skin candidiosis. Further tests proved that the bactericidal activity of TauBr is much stronger than TauCl especially at pH above 7, at which TauCl shows no bactericidal properties in subcytotoxic concentrations for mammalian cells, whereas TauBr destroys 99% of the bacteria tested. Variable susceptibility of the strains exposed to TauBr was found. TauCl shows no such selective bactericidal properties.

In order to conduct further tests to ascertain the antibacterial activity of the formulation containing taurine bromamine and prepared by the method described above, the imprinting method was chosen with use of Rodac plates with an area of 25 cm². For the purposes of study a Scheadler Agar Base medium produced by the firm Dico, catalogue number 212189, was used with addition of 5% ram blood. The medium was poured steadily onto the Rodac plates in aseptic conditions until a convex meniscus appeared. Before being applied, the medium was kept in enclosed conditions within a fridge at a temperature of ca. +4° C. and was used in a time not exceeding 7 days from the date of its preparation.

For the purpose of the studies several volunteers were selected—women aged between 23 and 27, who exhibited no discernable clinical pathological changes of the skin surface and who were not taking preparations with antibacterial activity during the studies, neither on the skin surface nor internally.

Tests for the antibacterial activity of taurine bromamine were conducted by performing 5 imprints of the dorsal skin on time at each of the patients examined. The skin area, on which the tests were to be performed, was determined in advance and marked with the numbers 1-5. After having marked the place where the test was to be performed, sterile gauze pads of area 4 cm² saturated with different substances were applied to the skin.

At the site marked 1—a gauze pad saturated with 0.9% inj. solution of NaCl (Polfa Lublin), which was a control substance without antibacterial activity was applied to the skin. At site 2—a gauze pad saturated with a 4-7 mM solution of taurine bromamine in a buffer with pH 5-pH 6.4 was applied to the skin. At site 3—a gauze pad saturated with a 4-7 mM solution of taurine bromamine in a buffer with pH 6.4-pH 7.4 was applied to the skin. At site 4—a gauze pad saturated with a 1% solution of salicylic acid in a buffer with pH 5 and at the site marked 5—a gauze pad saturated with a 1% solution of salicylic acid in a buffer with pH 7 was applied to the skin. 1% solutions of salicylic acid in buffers with pH 5 and pH 7 are substances which are used routinely in the treatment of acne. At all sites gauze pads were fixed firmly to the skin surface for 30 minutes and then were removed off. 5 minutes after their removal, as the skin was already dry, the Rodac type plates were pressed down so that the site treated with the substance tested was imprinted centrally on the plate. Next the plates were placed in anaerobic conditions using the Gas Pack system produced by the firm Biomerieux and incubated for 5 days at a temperature of 37° C. After completing the incubation the bacteria growths on the imprinting plates from sites 2-5 after treatment with the substance tested on each patient were compared with the control plate applied at site 1. Wherever possible, all the cultivated colonies of relatively aerobic and anaerobic bacteria were counted, to provide a figure for the concentration per cm² of the skin. The studies were repeated four times on the same patients at one-week intervals, which produced similar results. On the basis of the results obtained a considerable diminution in the number of bacteria on the skin area treated with the 4-7 mM solution of taurine bromamine in a buffer with pH 5-pH 7 was recorded for all the patients examined, and the biggest decrease in bacterial concentration, from the uncountable level of ca 10⁴ CFU/cm² to a level of 10¹-10² CFU/cm² was observed on the skin areas treated with a 5 mM solution of taurine bromamine in a buffer with pH 7. Decrease in bacterial concentration on a human skin was also observed when hands were washed with soap containing, for example bromamine taurine and glycerol or stearin.

Further studies of the antibacterial activity of taurine bromamine were conducted using a gel, a cream and a powder. In these tests, applications with a semisolid consistency and plastic features were made, called hereinafter, pharmaceutical or cosmetic preparations or compositions, particularly a taurine bromamine solution in an ethyl-cellulose gel with pH 5-pH 7, a cetomacrogol cream with pH 5-pH 7 and a powder suspension in a taurine bromamine solution.

The powder suspension in taurine bromamine solution was prepared on a base of liquid powder changing the proportions of the components by weight. The suspension consisted of, by weight: 14.0-60.0% of 10 mM Taurine bromamine solution, 10.0-24.0% of talc, 14.0-40.0% of glycerol, 5.0-30.0% of distilled water and 0.5-2.0% of menthol. The constituents of the suspension were stirred thoroughly until an homogenous dispersion of the solid phase was obtained.

The taurine bromamine solution in an ethyl-cellulose gel was prepared on a base of methyl-cellulose solution (Methocel 1500 cP) on a sterile phosphate buffer with pH 7.4 by pouring methyl cellulose to approx. one third of the volume of the prepared buffer solution at a temperature of ca. 90° C. and mixed so that the powder became completely moistened. The substance prepared in this way was cooled. The remainder of the buffer solution together with the taurine bromamine solution was added, similarly cooled, and was mixed thoroughly then left in a fridge to become clarified.

To prepare taurine bromamine solution in a cream form, (in brief—the preparation on a cream base), a cetomacrogol cream (Cetomacrogol Cream, Sorbolone Cream) was used according to The Extra Pharmacopoeia, Ed. 29. It was modified by introducing a phosphate buffer at pH 7.4 (with its composition KH₂PO₄ i Na₂HPO₄×12H₂O) in different amounts to replace distilled water. Next, the chloro-cresol in this cream was replaced with a mixture of preservative agents in different amounts. The mixture contained 1.0-3.0 g of benzyl alcohol, 120-200 mg of methyl-hydroxybenzoate or sodium hydroxybenzoesan and 60-100 mg of propyl-hydroxybenzoate (propyl hydroxybenzoesan). The production of the cream-based preparation began with melting the components of a cetomacrogol cream oil phase (emulsifying wax, liquid paraffin, white petroleum) in a water bath. Then the water phase (a sterile phosphate solution, a taurine bromamine solution, preserving agents) was emulsified in warmth at a temperature of ca. 60° C. into the oil phase and stirred until the mixture set.

The preparations based on a cream and gel base and the powder suspension were applied to the skin of the subjects at the places marked with numbers 1-5. Each of the preparations was applied to an area of ca. 4 cm² of every patient tested in the following way: at site “1”—a 0.4-0.8 mM taurine bromamine solution in a ethyl cellulose gel with pH 5-pH 7 was applied to the skin, at site “2”—a suspension of 3.0-4.5 mM taurine bromamine in an ethyl-cellulose gel with pH 5-pH 7, at site “3”—the preparation of 3.0-4.5 mM taurine bromamine solution in a cetomacrogol cream with pH 5-pH 7 and at site “4”—the powder suspension of 3.0-5.0 mM taurine bromamine was applied

The preparations were left on the skin area for 30 minutes and then the plates were pressed down so that the site exposed to the activity of the various substances was imprinted centrally on the plate. Later, as in case of the taurine bromamine solutions, the plates were placed in anaerobic conditions using the Gas Pack system produced by Firm Biomerieux and incubated for 5 days at a temperature of 37° C. After completing the incubation, the bacterial growth on the imprinting plates, from the sites marked by 1 to 5, which had been treated with the substance tested, were compared. Wherever it was possible, all cultivated colonies of relatively aerobic bacteria and anaerobic bacteria were counted, giving their occurrence per 1 cm² of the skin. The tests were conducted four times on the same people at one-week intervals, which produced similar results. On the basis of the results the most significant diminution in bacterial activity was observed in all patients on the skin areas treated with the suspension of above 3.5 mM taurine bromamine in the cetomacrogol cream at pH 7.

The taurine bromamine solutions in a cream and in a gel and powder suspensions in the taurine bromamine solutions, which may serve as examples of cosmetic compositions and pharmaceutical compositions, are given below. Proportions of the components of the substances mentioned below may be changed in response to the sensitivity of the skin. However, in case of a normal skin it is recommended that the molarity of these substances should not exceed 5 mM taurine bromamine, because above this level all the bacterial flora is destroyed, for example Staphylococcus epidermides.

### EXAMPLE 1

### 100 ml of 3.5 mM Modified Cream

| | | |
|---|---|---|
| Cetomakrogel emulsion | about | 15.0 |
| Liquid paraffin | about | 10.0 |
| Vaseline (or paraffin jelly) | about | 10.0 |
| Benzyl alcohol | | 1.5 |
| Sodium hydroxybenzoesan | | 0.15 |
| Ppropyl hydroxybenzoesan | | 0.08 |
| Propylene glycol | | 5.0 |
| 10 mM Taurine bromamine | | 35.0 |
| Water | ad | 100.0 |

### EXAMPLE 2

### 100 ml of 5.0 mM Modified Cream

| | | |
|---|---|---|
| Cetomakrogel emulsion | about | 15.0 |
| Liquid paraffin | about | 10.0 |
| Vaseline (or paraffin jelly) | about | 10.0 |
| Benzyl alcohol | | 1.5 |
| Sodium hydroxybenzoesan | | 0.15 |
| Propyl hydroxybenzoesan | | 0.08 |
| Propylglucose | | 5.0 |
| 10 mM Taurine bromamine | | 50.0 |
| Water | ad | 100.0 |

### EXAMPLE 3

### 100 ml of 3.5 mM Modified Gel

| | |
|---|---|
| 5% methylcellulose solution | 65.0 |
| 10 mM Taurine bromamine | 35.0 |

### EXAMPLE 4

### 100 ml of 5.0 mM Modified Gel

| | |
|---|---|
| 6.7% methylcellulose solution | 50.0 |
| 10 mM Taurine bromamine | 50.0 |

### EXAMPLE 5

### 100 ml of 3.5 mM Fluid Powder

| | | |
|---|---|---|
| 10 mM Taurine bromamine | | 35.0 |
| Talc | | 22.0 |
| Glycerol | | 38.0 |
| Menthol | | 1.0 |
| Distilled water | ad | 100.0 |

### EXAMPLE 6

### 100 ml of 5.0 mM Fluid Powder

| | | |
|---|---|---|
| 10 mM Taurine bromamine | | 50.0 |
| Talc | | 19.0 |
| Glycerol | | 29.0 |
| Menthol | | 1.0 |
| Distilled water | ad | 100.0 |

The preferred embodiments having been thus described, it will now be evident to those skilled in the art that further variation thereto may be contemplated. Such variations are not to be regarded as a departure from the invention, the true scope of the invention being set forth in the claims appended hereto.

What is claimed is:

1. A method for inhibiting pathogenic bacteria and fungi growth comprising
preparing a therapeutically effective amount of taurine bromamine [TauBr] or a solution with an effective quantity of taurine bromamine;
treating bacteria and fungi mycetes with the therapeutically effective amount of taurine bromamine [TauBr] or with the solution the effective quantity of taurine bromamine [TauBr].

2. The method according to claim 1 wherein
the effective quantity of taurine bromamine [TaurBr] is a solution of taurine bromamine at concentration 10-100% by weight.

3. A microbicidal composition containing
taurine bromamine [TauBr] being an essential component of the composition at concentration of 10-100% by weight.

4. The microbicidal composition according to claim 3 wherein
the taurine bromamine [TauBr] is a main ingredient of soap used for disinfecting a human skin.

5. The microbicidal composition according to claim 3 wherein
the taurine bromamine [TauBr] is used as a medicine in treatment of pathological dermal conditions, especially in treatment of acne.

6. The microbicidal composition according to claim 3 wherein
the taurine bromamine [TauBr] is used as a cosmetic in treatment of pathological dermal conditions, especially in treatment of acne.

7. The microbicidal composition according to claim 3 wherein
the taurine bromamine [TauBr] is a solution in an ethyl-cellulose gel with pH of 5-8.

8. The microbicidal composition according to claim 3 wherein
the taurine bromamine [TauBr] is a solution in a cream with pH of 5-8.

9. The microbicidal composition according to claim 3 wherein
the taurine bromamine [TauBr] is a solution in a powder with pH of 5-8.

10. The microbicidal composition according to claim 3 wherein
the taurine bromamine [TauBr] with glycerol constitutes a basis of cosmetic composition.

11. The microbicidal composition according to claim 3 wherein
the taurine bromamine [TauBr] with cetomakrogel emulsion constitutes a basis of cosmetic composition.

12. The microbicidal composition according to claim 3 wherein
the taurine bromamine [TauBr] with methylcellulose solution constitutes a basis of cosmetic composition.

13. The microbicidal composition according to claim 3 wherein
the taurine bromamine [TauBr] with glycerol constitutes a basis of pharmaceutical composition.

14. The microbicidal composition according to claim 3
the taurine bromamine [TauBr] with cetomakrogel emulsion constitutes a basis of pharmaceutical composition.

15. The microbicidal composition according to claim 3 wherein
the taurine bromamine [TauBr] with methylcellulose solution constitutes a basis of pharmaceutical composition.

16. The microbicidal composition according to claim 3 wherein the composition contains further talc in amount of 10.0-24.0% by weight, glycerol in amount of 14.0-40.0% by weight, distilled water in amount of 5.0-30.0% by weight and menthol in amount of 0.5-2.0% by weight.

17. The microbicidal composition according to claim 3 wherein the composition contains further 5% methylcellulose solution in amount of 50.0-65.0% by weight.

18. The microbicidal composition according to claim 3 wherein the composition contains further cetomakrogel emulsion in amount of about 15.0% by weight, liquid paraffin in amount of about 15.0% by weight, vaseline in amount of about 15.0% by weight and a mixture of preservative agents.

19. The microbicidal composition according to claim 3 wherein the composition contains further a lipophyllic phase—liquid paraffin, acetyl alcohol and an hydrophyllic phase—propylene glycol and estolate.
